(19) United States
(12) Patent Application Publication
(10) Pub. No.: US 2018/0221618 A1
(43) Pub. Date: Aug. 9, 2018

(54) Inhalation Therapy Device

(71) Applicant: Lunatussin, LLC, Folly Beach, SC (US)

(72) Inventors: Brandon Michael Strauss, Folly Beach, SC (US); David Strauss, Milton, GA (US); Jack Lindon, Milton, GA (US)

(21) Appl. No.: 15/888,012

(22) Filed: Feb. 3, 2018

Related U.S. Application Data
(60) Provisional application No. 62/454,105, filed on Feb. 3, 2017.

(51) Int. Cl. A61M 16/10; A62B 23/06; A61M 16/14; A61K 31/045; A61K 9/00

(52) U.S. Cl. CPC A61M 16/1045; A62B 23/06; A61K 9/0043; A61K 31/045; A61M 16/14

(57) ABSTRACT

A nasal respiratory delivery device includes fabric pads or fabric domes that cover the nostrils and includes a plastic strip with skin-sensitive adhesive for attachment of the device to the septum and the outside of each nostril, and provides for suppressing the human cough via moistening and warming inhaled air, thus soothing the respiratory passageways and mitigating the cough reflex. The nasal respiratory delivery device also provides for cough suppressants, nasal decongestants or other medical or breathing treatments. The nasal respiratory delivery device is placed across both nasal openings and adhered to the nasal septum and to the tissue surrounding the nostrils. The fabric pads or fabric domes are composed of nonwoven or woven manmade and/or natural materials that absorb and provide moisture or therapeutic agents during the act of breathing.

### CROSS-REFERENCE TO RELATED APPLICATIONS

This application claims the benefit of U.S. Provisional Application No. 62/454,105, entitled “Nasal Delivery System,” filed Feb. 3, 2017, which is incorporated herein by reference as if set forth herein in its entirety.

### BACKGROUND

This invention pertains to a nasal delivery device. More particularly, this invention pertains to an inhalation therapy device for providing moisture and/or volatile elements and compounds to the upper and lower respiratory system of an individual.

Humans suffer from numerous respiratory conditions ranging from severe diseases such as pneumonia, cystic fibrosis, obstructive sleep apnea (OSA), bronchitis, asthma, chronic bronchitis and emphysema (COPD) and sinusitis to less serious but debilitating conditions such as sinus congestion, chronic cough and nighttime cough. Patients with severe respiratory conditions are often treated through the administration of medications aerosolized by a nebulizer that allows the patient to inhale medication directly into the lungs. In other cases breathing gases, such as oxygen, supplied by respirators can be augmented with respiratory medications, again usually aerosolized by a nebulizer. When medication is delivered to the patient's respiratory system through a breathing tube, that breathing tube is often attached to the patient via a mask that covers either the nose or the mouth or both. Less severe respiratory conditions are often treated by passive administration of volatile compounds such as camphor and/or menthol, either supplied to the environment through a vaporizer or humidifier or by simple evaporation from the patient's body.

Clinically used volatile medications include various anesthetics (e.g., halothane, nitrous oxide, ether, and methoxyflurane), beta-adrenoreceptor agonist such as indacaterol for the treatment of COPD, corticosteroids such as predisone for the treatment of bronchitis and asthma, and amyl and butyl nitrates that act as vasodilators and muscle relaxants. Other respiratory medications include smelling salts (ammonium carbonate) used to arouse consciousness, dextromethorphan for cough suppression, decongestants such as phenylephrine and antihistamines such as diphenhydramine and doxylamine. Sinus congestion and cough are often treated with eucalyptus oil, menthol and camphor that are commonly found in over-the-counter medications. In addition to these volatile pharmaceutical medications, numerous essential oils have been employed and are claimed to combat ailments including congestion (cedarleaf oil), headache (peppermint oil), insomnia and muscle spasms (sandlewood oil) and indigestion (frankincense oil and wintergreen oil). These are non-prescription and homeopathic remedies commonly referred to as aromatherapy agents. These various aromatherapy treatments are generally delivered via diffusers (U.S. Pat. No. 5,805,768 and U.S. Pat. No. 6,041,503), inhalers (U.S. Pat. No. 7,997,280) or scented candles.

Many clinical artificial respirators and continuous positive airway pressure (CPAP) machines augment the humidity of the air supplied to the patient. For example, U.S. Pat. No. 9,855,398 details the use of heaters to control the humidity of breathable gas supplied by respiratory ventilation systems and CPAP machines. U.S. Pat. Nos. 8,678,355 and 9,849,258 describe devices for humidifying the breathing air of artificial respirators in which a water-filled container provides for air from the respirator to be passed through the high humidity chamber before delivery to the patient.

Often simply increasing the humidity of the patient's environment through the use of a humidifier or vaporizer can ameliorate the conditions of chronic or nighttime cough. Increases in humidity levels can be accomplished in numerous ways. Many humidifying devices simply employ liquid water that is allowed to evaporate. Others facilitate the conversion of water from the liquid to the gaseous state using heaters (see above) or devices such as ultrasonic transducers (U.S. Pat. Nos. 9,353,960; 9,004,065 and 8,800,970).

Many uses for humidifiers have been identified. Patents have issued for humidifiers designed to augment the humidity of living spaces. These often take the form of either freestanding “room” humidifiers or vaporizers (U.S. Pat. Nos. 9,752,790 and 6,226,451) or devices incorporated directly into a building's heating equipment or units placed over heating vents to supply moisture to the heated air (U.S. Pat. Nos. 4,834,285; 8,794,601; 8,905,384 and 9,476,604). Other uses for humidifiers include humidors for storing cigars and other tobacco products (U.S. Pat. Nos. 4,428,892 and 5,829,452) or humidifiers designed to protect wooden musical instruments (U.S. Pat. Nos. 7,892,327 and 8,220,782).

Several issued patents have introduced the concept of a personal humidifier. Often these are simply small versions of humidifier that can be transported by the user (U.S. Pat. Nos. 5,673,360 and 9,845,962). In other cases devices have been patented that provide humid air to an individual through an appliance attached to the nose or through a facemask covering the nose and the mouth. U.S. Pat. No. 6,691,706 describes a hollow headset that contains water and is contiguous with a wicking material that is positioned under the user's nose and supplies moisture to the user's nasal passages. U.S. Pat. No. 5,373,841 claims a nasal humidifier that consists of a container that holds heated water and a conduit with a nasal adapter that delivers humidified air to the nostrils. U.S. Pat. No. 4,941,467 describes a humidification facemask that contains a moisturizing pad that humidifies the air breathed by the user.

Several different media have been used to provide and control moisture in humidifiers and in other applications. As described above, many humidifiers simply employ water-filled chambers, with the air in these chambers humidified by evaporation. Other devices use sponge-like materials such as hydrophilic florist foam (a chemically-modified, hydrophilic Styrofoam). This material absorbs water, preventing it from leaking from the humidifier, and allows the absorbed water to evaporate slowly as described in U.S. Pat. Nos. 5,127,184 and 6,178,688.

Other devices employ various hygroscopic compounds, including humectants, to control moisture levels. Humectants are natural or synthetic substances (both solid and liquid) that promote the absorption of moisture from the air. These compounds have numerous commercial applications in foods (for improved texture and taste), cosmetics (for moisturizing skin and hair), and medical, agricultural and tobacco products (for moisture control). U.S. Pat. No. 4,559,950 describes a diagnostic electrode that uses humectants to control moisture levels. U.S. Pat. No. 5,865,869 describes the use of solutions containing a humectant (such as sorbitol), along with a wetting agent and a binding agent, to improve the water retention by plant roots. Humectants have also been used to control humidity levels in humidors (U.S. Pat. Nos. 4,428,892; 7,892,327 and 8,220,782).

Other forms of hygroscopic materials used to retain and control moisture include numerous fibers, both natural plant-based fibers (such as cotton, cellulose, coco, jute, ramie and sisal fibers) and animal-based fibers (such as wool, mohair, alpaca, angora and lama fibers). Other hygroscopic fibers include man-made fibers, such as rayon and viscose, produced by chemical modification of natural, plant-based fibers such as cellulose.

Synthetic hydrogels are also employed to control moisture levels. Synthetic hydrogels are hydrophilic polymers that readily absorb water. Man-made hydrogels are often synthesized from polyols such as poly(vinyl alcohol), poly(vinylpyrrolidone), poly(ethylene glycol) or polyacrylamide. One of the most common synthetic hydrogels is synthesized from poly(acrylic acid) and is commonly referred to as superabsorbent (SA). This material can hold up to 200 times its weight in water. The most common form of superabsorbent is superabsorbent particles (SAP). SAP is often used in personal care products such as diapers. Acrylic-based superabsorbents have also been manufactured in the form of fibers (U.S. Pat. Nos. 5,582,786 and 6,436,323). Other synthetic, water-absorbent fibers have been manufactured using polymers such as polyacrylonitrile (U.S. Pat. No. 4,873,143) and maleic anhydride (U.S. Pat. No. 4,743,244). These and other manufactured water absorbent fibers have been used for medical application (such as wound care and transdermal delivery), agricultural application (such as fabric for hydration control and plant transport), hygiene products (such as disposable diapers and feminine hygiene products) and apparel for control of body moisture and cooling. U.S. Pat. No. 9,597,234 claims an absorbent wound dressing that contains superabsorbent fibers.

Two devices utilize a flexible, conformable strip and a skin-compatible adhesive attached to the lower portion of the nose, with a filter or valve covering the nostrils. These devices exist to solve different problems than the present invention. First Defense Nasal Screens (described in U.S. Pat. Nos. 8,550,079 and 9,132,300) attach to the nose in a similar manner and operate to filter out allergens and pollutants. Theravent strips (described in U.S. Pat. Nos. 8,302,607 and 8,985,116) are marketed for the prevention of snoring and to treat sleep apnea. Both of these devices are held to the nasal openings by adhesive coatings on the devices; neither is designed to provide for the inhalation of volatile compounds.

Numerous devices are described in the patent literature that provide volatile compounds, other than water vapor, to the respiratory tract of the user. These include active respiratory therapy devices (U.S. Pat. Nos. 8,534,284; 8,678,355 and 9,802,023) and nebulizers, atomizers, diffusers and inhalers (U.S. Pat. Nos. 8,915,245 and 9,597,477). Many of these devices use heat to volatilize liquid compounds. Others use mechanical means or surface acoustic waves to produce liquid droplets, normally in the range of 1 to 10 micrometers in diameter.

### BRIEF SUMMARY

According to one embodiment of the present invention, an inhalation therapy device having a breathable fabric pad covering both nostrils, a flexible base strip with a skin-sensitive adhesive for attaching the device to the septum and the outside edges of each nostril, is provided. The breathable fabric pad absorbs moisture from exhaled air and provides that moisture into the nasal passages during inhalation.

When applied to the user, the nasal delivery system suppresses the human cough by capturing moisture from exhaled air and moistening and warming inhaled air, thus soothing the irritated respiratory passageways and mitigating the cough reflex. The nasal delivery strip is placed over both nasal openings and is adhered to the septum and the left and right sides of the nose (the inferior columella, the alar rim and the nasal facet), with the fabric pad placed so as to cover both nostrils. The fabric pad is comprised of nonwoven or woven manmade and/or natural materials that absorb and release moisture during the act of breathing. In another embodiment, the fabric pad contains and delivers aromatherapy compounds, cough suppressants and nasal decongestants or other breathing medications.

In another embodiment, the nasal delivery system includes a pair of fabric domes placed along a center section of a flexible base strip, which includes an adhesive layer on the side from which the fabric domes protrude. The flexible base strip is made from a flexible plastic or fabric and is conformable to the skin and is coated with a skin-sensitive adhesive.

In various embodiments, the fabric pad or fabric domes provide for the absorption of moisture into the particular fabric or fabric coatings for alleviating dry air effects to minimize or eliminate dry air cough.

In other embodiments, the fabric pad or fabric domes are a nonwoven manmade material. In another embodiment, the fabric pad or fabric domes are woven manmade materials. In another embodiment, the fabric pad or fabric domes are nonwoven natural materials. In another embodiment, the fabric pad or fabric domes are woven natural materials. In yet other embodiments, the fabric pad or fabric domes includes sodium polyacrylate fibers or similar compounds, and/or naturally occurring substances that absorb and give up moisture.

In other embodiments, the fabric pads or domes are coated with humectants, compounds that provide for the absorption of moisture into the fabric domes from the exhaled air. The moistened fabric pad or fabric domes provide moisture to inhaled air, alleviating irritation of the nasal passages to minimize or eliminate coughing exacerbated by dry air.

In other embodiments, the fabric pad or fabric domes are coated with menthol, eucalyptus, or other known natural cough suppressants or aroma therapy agents which, activated by the warmth of exhaled air, vaporize and permeate inspired air as it enters the nasal passages. In yet another embodiment, the fabric pad or fabric domes include microencapsulated menthol, eucalyptus, or other known natural cough suppressants or aromatherapy agents activated by the warmth of exhaled air.

In other embodiments, the fabric pad or fabric domes do not include sodium polyacrylate or other similar compounds, or natural occurring substances that absorb and give up moisture. Such an embodiment relies on manmade or naturally occurring materials and fibers to capture warm, exhaled moisture, subsequently moisturizing and warming air upon inhalation.

Other systems, methods, features and advantages of the present invention will be or become apparent to one with skill in the art upon examination of the following drawings and detailed description. It is intended that all such additional systems, methods, features and advantages be included within this description and be within the scope of the present disclosure.

### BRIEF DESCRIPTION OF THE DRAWINGS

The above-mentioned features will become more clearly understood from the following detailed description read together with the drawings in which:

FIG. 1 is a top view of a double nostril nasal delivery device;

FIG. 2 is an exploded perspective view of the nasal delivery device of FIG. 1;

FIG. 3 is a top view of an alternative nasal delivery device;

FIG. 4 is a side view of the alternative nasal delivery device of FIG. 3;

FIG. 5 is an exploded perspective view of the alternative nasal delivery device of FIG. 3;

FIG. 6 is a top view of a second alternative nasal delivery device;

FIG. 7 is an exploded perspective view of the second alternative nasal delivery device of FIG. 6;

FIG. 8 is a partially exploded perspective view of the second alternative nasal delivery device of FIG. 6;

FIG. 9 is a top view of a third alternative nasal delivery device;

FIG. 10 is an exploded perspective view of the third alternative nasal delivery device of FIG. 9;

FIG. 11 is a partially exploded perspective view of the third alternative nasal delivery device of FIG. 9;

FIG. 12 is a top view of a single nostril nasal delivery device;

FIG. 13 is a side view of the single nostril nasal delivery device of FIG. 12;

FIG. 14 is an exploded perspective view of the single nostril nasal delivery device of FIG. 12;

FIG. 15 is a perspective view of the single nostril nasal delivery device of FIG. 12;

FIG. 16 is a frontal view of a face illustrating application of one embodiment of the nasal delivery device;

FIG. 17 is a frontal view of a face illustrating an embodiment of the nasal delivery device attached to the nose;

FIG. 18 is a first graph demonstrating the capability of fabrics that include superabsorbent fibers to capture moisture and enhance the humidity of inhaled air;

FIG. 19 is a second graph demonstrating the capability of fabrics that include superabsorbent fibers to capture moisture and enhance the humidity of inhaled air;

FIG. 20 is a third graph demonstrating the capability of fabrics that include superabsorbent fibers to capture moisture and enhance the humidity of inhaled air; and

FIG. 21 is a graph demonstrating the capability of a fabric with superabsorbent fibers to maintain a stable level of inhaled air humidity over a 5-hour time period.

### DETAILED DESCRIPTION

An inhalation therapy device and method are disclosed. The inhalation therapy device, also referenced as a nasal delivery device, includes a fabric pad of a breathable fabric layer attached to a base strip via adhesive and secured to the nose of an individual. The nasal delivery device is applied over one or both nostril openings to provide moisture and/or volatile elements and compounds to the upper and lower respiratory system of an individual.

The nasal delivery device is a wearable, personal respiratory delivery system used to deliver moisture, volatile medications and/or aromatherapy agents to the sinuses, pharynx, bronchial tree and lungs via inhalation.

The nasal delivery device provides for the slow, continuous administration of volatile elements and compounds to the upper and lower respiratory system of individuals. The nasal delivery device provides for a porous, highly permeable and breathable material and an adhesive that allows the device to be adhered to the lower portion of the nose covering the nostril openings (the external naris) and provides for easy removal. In one embodiment, the highly permeable material is comprised of hygroscopic fibers or humectant-coated fibers, to capture moisture from exhaled breath, and delivers the resulting moisture to the inhaled air. In another embodiment, volatile elements and compounds are incorporated into the device, coating the breathable material. The volatile compounds readily evaporate or sublimate at the temperatures and pressures found in the user's environment and are inhaled into the user's respiratory system.

In one embodiment, the nasal delivery device covers each nostril with a fabric pad affixed to a plastic film and secured to the nose by a skin-sensitive adhesive. The nasal delivery device that delivers moisture is particularly effective for alleviating both daytime and nighttime cough, as well as for irritations that affect the nasal passages and/or throat, such as dry nasal passages, burning nasal passages, inflamed nasal passages, and similar throat irritations.

When applied, the nasal delivery device suppresses the human cough via moistening and warming inhaled air, thus soothing irritated respiratory passageways and mitigating the cough reflex. In this particular embodiment, the nasal delivery device is placed over both nostrils and is adhered to the columella (the inferior margin of the septum), the alar ridge (the left and right sides of the nostrils at the end of the nose) and the nasal facet (the tissue between the upper lip and the nose), with the fabric pad covering both nostril openings. The fabric pad provides for absorbing and delivering moisture and for other agents to alleviate the cough reflex, whether for dry cough, daytime cough, or nighttime cough. Additionally, the fabric pad is also utilized to contain and deliver aromatherapy compounds, cough suppressants and nasal decongestants, as well as other medical or breathing treatments.

One particular application of the invention is portable, disposable, respiratory therapy devices that provide for an increase in the water vapor content of the air entering the nasal passages during normal inhalation. This application has the potential to ameliorate a condition known as nighttime cough, caused and exacerbated by low humidity of the air in the sleep environment. Two primary modes that provide for this increase in humidity are the use of humectant coatings on the breathable fabrics of the device or the incorporation of superabsorbent fibers within the breathable fabric structure.

Typical breathable fabrics for these applications are low density, open nonwoven structures such as spun-laced (hydroentangled) or needle-punched fabrics. These fabrics are coated with humectants (hygroscopic compounds) such as sugar alcohols (glycerol, sorbitol, maltitol, etc.), glycols (propylene glycol, butylene glycol, etc.) or even naturally occurring compounds such are Aloe Vera gel and honey. Such coated, breathable fabrics are either pre-moistened to supply moisture to the air inhaled through the device or supplied without pre-moistening. When supplied without pre-moistening, the humectant coating on the device captures and retains the moisture available in the high-humidity exhaled air that is then released during inhalation, increasing the humidity of the inhaled air.

Breathable nonwoven materials are also available that contain superabsorbent and other hygroscopic fibers. Superabsorbents and superabsorbent fibers are water-absorbing polymers generally composed of sodium polyacrylates. These compounds are hygroscopic materials that can absorb and release water molecules to control humidity levels. Both of these approaches, using humectant coatings and fabrics that contain superabsorbent fibers, have proven to be highly effective means of increasing the water vapor content of the inhaled air as shown in the examples below.

Another application of the present invention provides the capability to supply volatile compounds, other than water vapor, to the respiratory tract of the user. Many devices use heat to volatilize liquid compounds, while others use mechanical means or surface acoustic waves to produce liquid droplets, normally in the range of 1 to 10 micrometers in diameter. In contrast, the nasal delivery devices disclosed herein rely on the normal volatilization properties of compounds, enhanced by the elevated temperatures present at the nasal orifice. These naturally volatile compounds provide gaseous molecules, either by sublimation (solid compounds) or evaporation (liquids), to the respiratory system of the wearer through simple inhalation. Examples of such compounds range from aromatherapy compounds (such as wintergreen, spearmint, and tea tree oils) and common cough remedies (such as camphor, menthol and eucalyptus oil) to decongestants and antihistamines (such as phenylephrine and diphenhydramine). The present invention provides a means to deliver such volatile compounds in a low-dose, continuous mode using a wearable, disposable, lightweight, inconspicuous device.

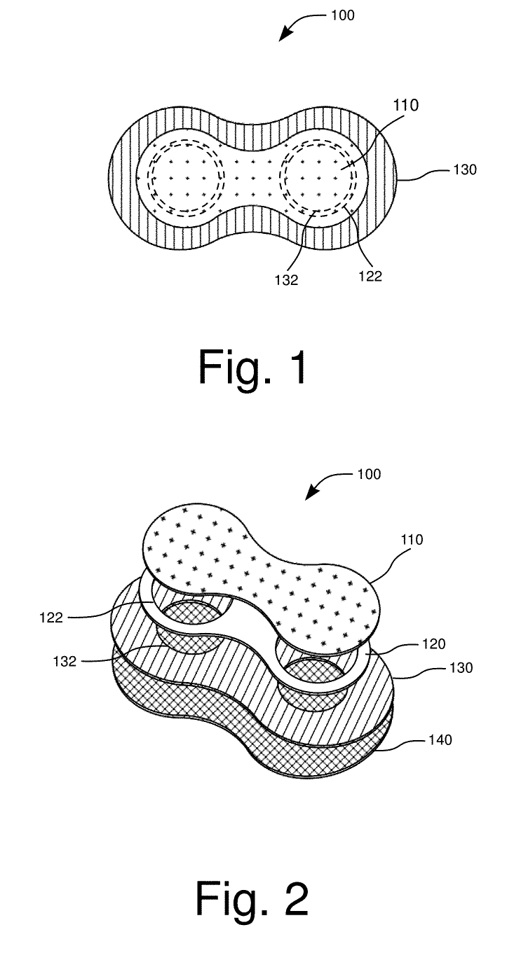

FIG. 1 is a top view of a double nostril nasal delivery device 100, and FIG. 2 is a perspective view of the nasal delivery device 100. In the illustrated embodiment, the nasal delivery device 100 includes an hourglass shaped fabric pad 110 placed lengthwise along a center section of a flexible base strip 130 and held in place by a thin, flexible double-sided adhesive layer 120. A typical flexible base strip 130 also includes an adhesive layer, applied to the backside (or underside not shown in FIG. 2) of the flexible base strip 130, and provides for affixing the nasal delivery device 100 to the nose. Prior to application by the user, the skin-sensitive adhesive layer on the flexible base strip 130 is typically covered by a removable release paper layer 140.

In one typical embodiment, the flexible base strip 130 is made from a flexible and conformable plastic or fabric strip. The flexible base strip 130 is flexible enough to conform to one side of the nose, extend across the septum, and onto the other side of the nose. In various embodiments, the flexible base strip 130 may be made from flexible plastic film and other such like materials that provide for flexibility and durability. In the illustrated embodiment, the adhesive layer of the flexible base strip 130 is a skin-sensitive material that provides for adhering to the skin without causing irritation.

A double-sided adhesive layer 120 adjoins the flexible base strip 130 to the fabric pad 110. The double-sided adhesive layer 120, the flexible base strip 130, and the fabric pad 110 have an hourglass shape. Both the double-sided adhesive layer 120 and the flexible base strip 130, have a pair of adhesive openings 122 and base strip openings 132 respectively, which align with the nostril openings when the nasal delivery device 100 is attached to the nose.

The fabric pad 110 is an hourglass-shaped fabric layer situated in a center section of the flexible base strip 120. The fabric pad 110 is positioned to cover each nostril when attached. In one embodiment, the fabric pad 110 includes specific fibers or fiber coatings that collect moisture from exhaled air and delivers moisture to air inhaled into the nostrils during normal breathing.

In another embodiment, a humectant substance coated onto the fabric pad material is utilized to collect moisture and keep the fabric material moist. Specifically, a humectant attracts water vapor in the exhaled air by absorption and delivers that moisture to the inhaled air.

In another embodiment, sodium polyacrylate fibers in the fabric pad are utilized to absorb water vapor into the fabric and release that moisture into the inhaled breath. Sodium polyacrylate has the capability to absorb up to 200 times its mass in water.

In other embodiments, other natural, hygroscopic fibers, such as cellulose, cotton, wool and silk are used to absorb moisture from the exhaled breath and deliver that moisture to the inhaled breath. In still other embodiments, other hygroscopic natural plant-based fibers, such as hemp and jute or animal-based hygroscopic fibers, such as mohair and alpaca, or man-made hygroscopic fibers, such as rayon, are used.

In various embodiments, humectants, sodium polyacrylate, and other such materials are utilized together to provide for absorbing moisture into the fabric pad or domes, so that such moisture, once inhaled, provides for relief of coughing. Such embodiments also reduce coughing exacerbated by dry air, for example.

In one embodiment, the fabric pad 110 is a non-woven, manmade material. In another embodiment, the fabric pad 110 is woven, manmade material. In another embodiment, the fabric pad 110 is a nonwoven, natural material such as a felt material. In another embodiment, the fabric pad 110 is a woven, natural material, such as a cotton cloth. In yet other embodiments, the fabric pad 110 includes sodium polyacrylate fibers or similar compounds, and/or naturally occurring substances that absorb and give up moisture.

In another embodiment, the fabric pad 110 is coated with camphor, menthol, eucalyptus, or other known natural cough suppressants that vaporize and permeate inhaled air as it enters the nasal passages. In another embodiment, the fabric pad 110 includes microencapsulated menthol, eucalyptus, or other known natural cough suppressants that are activated by the warmth of exhaled air. In another embodiment, the fabric pad 110 is treated with aromatherapy agents. In other embodiments, the fabric pad is 110 treated with other synthetic or natural compounds that are microencapsulated.

In some embodiments, the fabric pad 110 is comprised of multi-layer materials wherein any particular layer contributes to various methods of moisture retention and delivery for reducing or eliminating the effects of dry air, while other layers provide therapeutic compounds such as aromatherapy compounds, cough suppressants, nasal decongestants, or other medical or breathing treatments.

The double nostril nasal delivery device 100 is affixed to the nose by first centering the fabric pad 110 over the nostrils and then pressing the center of the flexible base strip 130 firmly against the nasal septum. The flexible base strip 130 is then pressed and affixed to the nasal tissue surrounding the nasal openings.

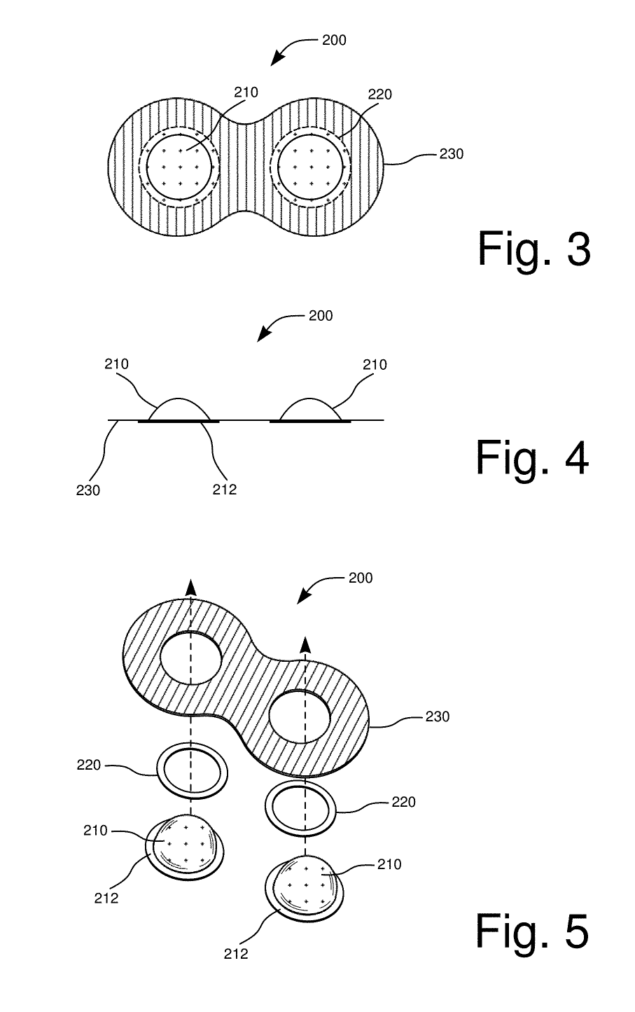

FIG. 3 is a top view of an alternative embodiment of a double nostril nasal delivery device 200, FIG. 4 is a side view of the alternative embodiment of a double nostril nasal delivery device 200, and FIG. 5 is a perspective view of the alternative embodiment of the nasal delivery device 200. In the illustrated embodiment, the nasal delivery device 200 includes a pair of fabric domes 210 that protrude through respective openings in the flexible base strip 230. The fabric domes 210 are produced by sealing (either by heating or with adhesive) circular fabric pads around their perimeters. This sealing produces a flat perimeter 212 at the base of the respective fabric dome 210. The fabric domes 210 extend through the openings of the flexible base strip 230 and are attached via double-sided adhesive rings 220. The flexible base strip 230 incorporates a skin-sensitive adhesive layer, affixed to the topside (or skin-side) of the flexible base strip 230, to adhere the nasal delivery device 200 to the nose. Prior to application by the user, the skin adhesive layer on the flexible base strip 130 is typically covered with a removable release paper layer (not shown).

In one typical embodiment, the flexible base strip 230 is made from a flexible and conformable plastic or fabric strip. The flexible base strip 230 is flexible enough to conform to one side of the nose, extend across the septum, and onto the other side of the nose. In various embodiments, the flexible base strip 230 may be made from flexible plastic film and other such like materials that provide for flexibility and durability. In the illustrated embodiment, the adhesive layer of the flexible base strip 230 is a skin-sensitive material that provides for adhering to the skin without causing irritation.

The fabric dome 210 is positioned to cover each nostril when attached. In one embodiment, the fabric dome 210 includes specific fibers or fiber coatings that collect moisture from exhaled air and delivers moisture to air inhaled into the nostrils during normal breathing.

In various embodiments, substances such as a humectant substance, or sodium polyacrylate fibers, are coated onto the fabric pad material is utilized to collect moisture and keep the fabric material moist and release that moisture into the inhaled breath. Specifically, a humectant attracts water vapor in the exhaled air by absorption and delivers that moisture to the inhaled air. Sodium polyacrylate has the capability to absorb up to 200 times its mass in water.

In other embodiments, other natural, hygroscopic fibers, such as cellulose, cotton, wool and silk are used to absorb moisture from the exhaled breath and deliver that moisture to the inhaled breath. In still other embodiments, other hygroscopic natural plant-based fibers, such as hemp and jute or animal-based hygroscopic fibers, such as mohair and alpaca, or man-made hygroscopic fibers, such as rayon, are used.

In various embodiments, humectants, sodium polyacrylate, and other such materials are utilized together to provide for absorbing moisture into the fabric domes 210, so that such moisture, once inhaled, provides for relief of coughing. Such embodiments provide for reducing coughing exacerbated by dry air, for example.

In one embodiment, the fabric domes 210 are a non-woven, manmade material. In another embodiment, the fabric domes 210 are woven, manmade material. In another embodiment, the fabric domes 210 are a nonwoven, natural material such as a felt material. In another embodiment, the fabric domes 210 are a woven, natural material, such as a cotton cloth. In yet other embodiments, the fabric domes 210 include sodium polyacrylate fibers or similar compounds, and/or naturally occurring substances that absorb and give up moisture.

In another embodiment, the fabric domes 210 are coated with camphor, menthol, eucalyptus, or other known natural cough suppressants that vaporize and permeate inhaled air as it enters the nasal passages. In another embodiment, the fabric domes 210 include microencapsulated menthol, eucalyptus, or other known natural cough suppressants that are activated by the warmth of exhaled air. In another embodiment, the fabric domes 210 are treated with aromatherapy agents. In other embodiments, the fabric domes 210 are treated with other synthetic or natural compounds that are microencapsulated.

In some embodiments, the fabric domes 210 include multi-layer materials so that any particular layer contributes to various methods of moisture retention and delivery for reducing or eliminating the effects of dry air, while other layers provide therapeutic compounds such as aromatherapy compounds, cough suppressants, nasal decongestants, or other medical or breathing treatments.

The double nostril nasal delivery device 200 is affixed to the nose by first centering the fabric domes 210 over the nostrils and then pressing the center of the flexible base strip 230 firmly against the nasal septum. The flexible base strip 230 is then pressed and affixed to the nasal tissue surrounding the nasal openings.

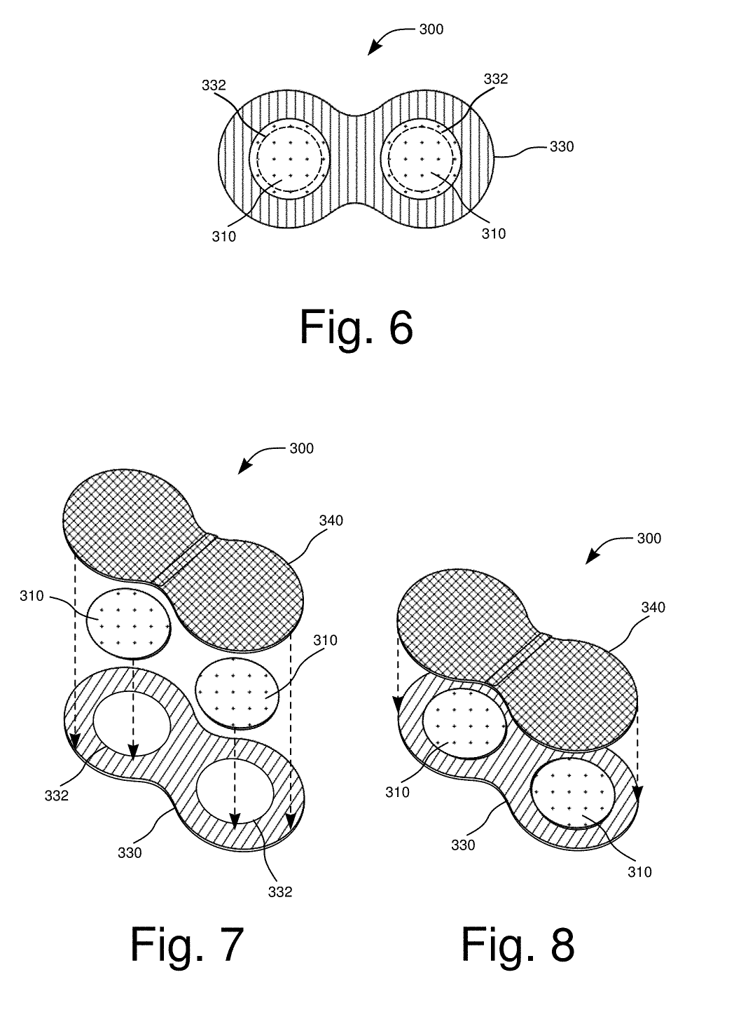

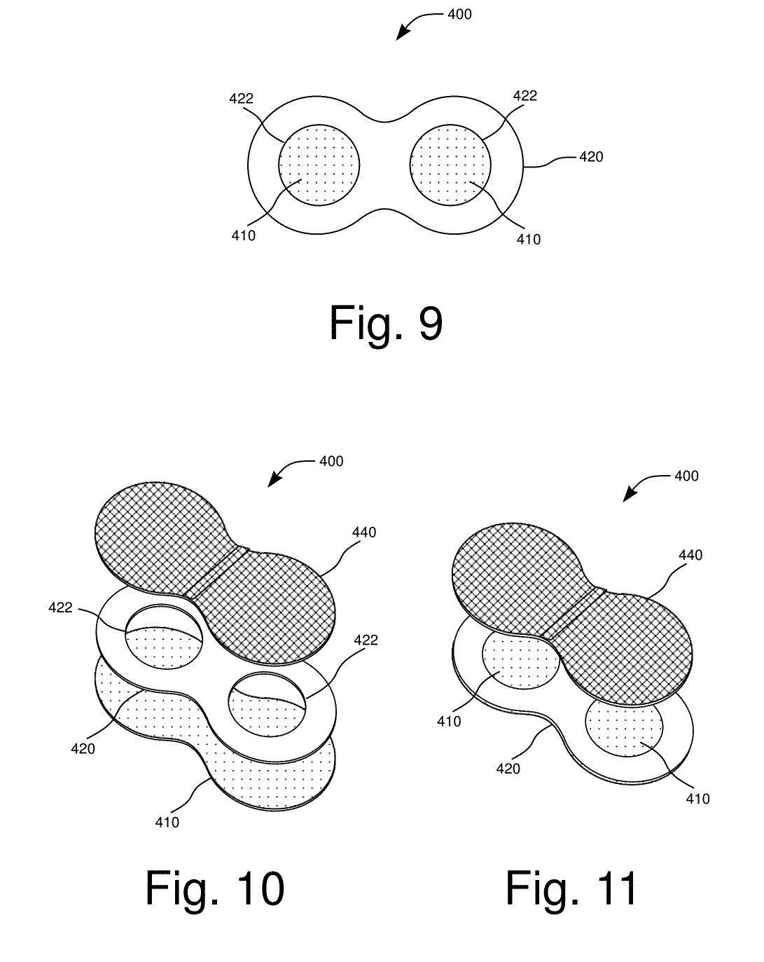

FIG. 6 is a top view of a second alternative embodiment of a double nostril nasal delivery device 300, FIG. 7 is an exploded perspective view of the second alternative embodiment of the nasal delivery device 300, and FIG. 8 is a partially exploded perspective view of the second alternative embodiment of the nasal delivery device 300.

The second alternative embodiment of the double nostril nasal delivery device 300 utilizes circular fabric pads 310 attached to an adhesive side of a flexible adhesive base strip 330. The circular fabric pads 310 are affixed to cover the circular openings 332 of the flexible adhesive base strip 330. The flexible adhesive base strip 330 includes a skin-sensitive adhesive layer on one side. The skin-sensitive adhesive layer serves a dual purpose of securing the circular fabric pads 310 to the flexible adhesive base strip 330 and also securing the nasal delivery device 300 to the nose. Prior to application by the user, the skin adhesive layer on the flexible adhesive base strip 330 is covered with a removable release paper layer 340.

In one typical embodiment, the flexible base strip 330 is made from a flexible and conformable plastic or fabric strip and is flexible enough to conform to one side of the nose, extend across the septum, and onto the other side of the nose. In various embodiments, the flexible base strip 330 may be made from flexible plastic film and other such like materials that provide for flexibility and durability. In the illustrated embodiment, the adhesive layer of the flexible base strip 330 is a skin-sensitive material that provides for adhering to the skin without causing irritation.

The circular fabric pads 310 are positioned to cover each nostril when attached and include specific fibers or fiber coatings that collect moisture from exhaled air and deliver moisture to air inhaled into the nostrils during normal breathing.

The double nostril nasal delivery device 300 is affixed to the nose by first centering the circular fabric pads 310 over the nostrils and then pressing the center of the flexible base strip 230 firmly against the nasal septum. The flexible base strip 330 is then pressed and affixed to the nasal tissue surrounding the nasal openings.

FIG. 9 is a top view of a third alternative embodiment of a double nostril nasal delivery device 400, FIG. 10 is an exploded perspective view of the third alternative embodiment of the nasal delivery device 400, and FIG. 11 is a partially exploded perspective view of the third alternative embodiment of the nasal delivery device 400. The third alternative nasal delivery device 400 includes a double-sided flexible adhesive strip 420 and a fabric pad 410. In the illustrated embodiment, the fabric pad 410 and the flexible adhesive strip 420 are of an hourglass shape. The flexible adhesive strip 420 includes a pair of circular openings 422 that align with the nostrils when attached to the nose.

In the illustrated embodiment, the flexible adhesive strip 420 includes adhesive on both sides. The flexible adhesive strip 420 includes a skin-sensitive adhesive layer, on one side for securing the nasal delivery device 400 to the nose. The fabric pad 410 is affixed to the nasal delivery device 400 via adhesive on the second side.

Prior to application by the user, the skin adhesive layer on the flexible base strip 420 is covered with a removable release paper layer 440.

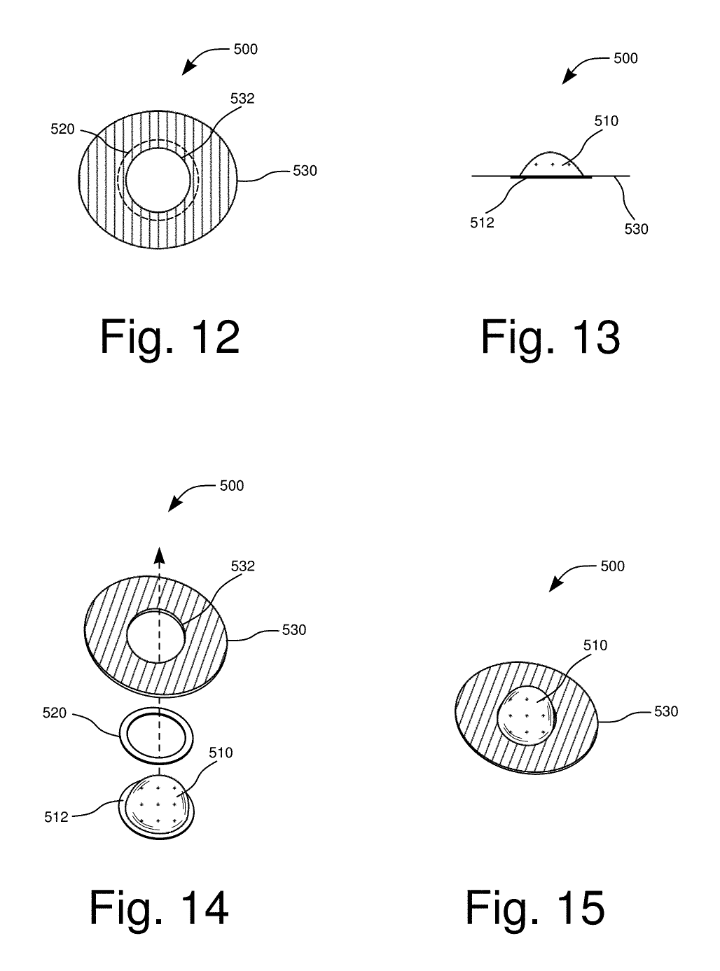

FIG. 12 is a top view of a single nostril nasal delivery device 500, FIG. 13 is a side view of the single nostril nasal delivery device 500, FIG. 14 is an exploded perspective view of the single nostril nasal delivery device 500, and FIG. 15 is a perspective view of the single nostril nasal delivery device 500. The single nostril nasal delivery device 500 includes a single fabric dome 510. The fabric dome 510 is produced by sealing (either by heating or with adhesive) circular fabric pads around its perimeter. This sealing produces a flat perimeter 512 at the base of the fabric dome 510. The fabric dome 510 is attached at a single opening 532 centered within an oval-shaped, flexible base strip 530. The flexible base strip 530 includes a skin-sensitive adhesive (on the skin side layer) for securing the single nostril nasal delivery device 500 to the nose. The fabric dome 510 is affixed via the flat perimeter 512 and an adhesive ring 520 to the underside of the flexible base strip 530, and the fabric dome 510 protrudes through the single opening 532. The flexible base strip 530 includes a skin-sensitive adhesive on the upper side for securing the single nostril nasal delivery device 500 to the nose. Prior to application by the user, the skin side adhesive layer on the flexible base strip 530 is typically covered with a removable release paper layer (not shown).

The single nostril nasal delivery device 500 provides a single fabric dome 510 for alleviation of cough or nasal congestion where the effects are predominantly needed within a single nostril. For example, the single nostril delivery device 500 is utilized to deliver a decongestant or other material within a single nostril. Alternatively, the single nostril delivery system can be used in pairs.

The flexible base strip 530 includes an adhesive layer on the side of the flexible base strip 530 from which the fabric dome 510 protrudes. The adhesive layer on the flexible base strip 530 is a skin-sensitive material that provides for adhesion to the skin without causing irritation.

The fabric dome 510 is oriented to protrude into a single nostril. As with the fabric pads above, the fabric dome 510 provides for the absorption of moisture onto the particular fabric. As above, a humectant can be utilized to keep the fabric material moist. Water vapor is drawn into the humectant on the fabric surface. In another embodiment, sodium polyacrylate fiber is utilized to absorb moisture into the fabric. In various embodiments, humectants, sodium polyacrylate, and other such materials are utilized together to absorb moisture into the fabric dome, so that such moisture provides for relief of coughing. Such embodiments provide for alleviating coughing problems by alleviating dry air effects to minimize or eliminate dry air cough.

As with the fabric pads described above, and in various embodiments, the fabric dome 510 is a nonwoven manmade material, a woven manmade material, a nonwoven natural material, or a woven natural material. In yet other embodiments, the fabric dome 510 includes sodium polyacrylate or similar compounds, and/or naturally occurring substances that absorb and give up moisture.

In another embodiment, the fibers of the fabric dome 510 are coated with menthol, eucalyptus or other known natural cough suppressants. In other embodiments, the fabric dome 510 fibers include microencapsulated menthol, eucalyptus, or other known natural cough suppressants activated by the warmth of exhaled breath. In other embodiments, the fabric dome 510 is treated with synthetic or natural compounds that are microencapsulated.

In yet another embodiment, the fabric dome 510 does not include sodium polyacrylate or other similar compounds that absorb and give up moisture. Such embodiments rely on naturally occurring materials and fibers to capture exhaled warm moisture, subsequently moisturizing and warming the air upon inhalation.

In some embodiments, the fabric dome 510 is comprised of multi-layer materials, wherein any particular layer contributes to various moisture retention and/or other methods for reducing or eliminating the effects of dry air or providing cough suppressant medication, while other layers provide therapeutic compounds such as aromatherapy compounds, cough suppressants, nasal decongestants, or other medical or breathing treatments.

The single nostril nasal delivery system 500 is affixed to the nose by first centering the fabric dome 510 over the nostril and then pressing the flexible base strip 520 firmly against the nasal septum and to the nasal tissue surrounding the nasal openings.

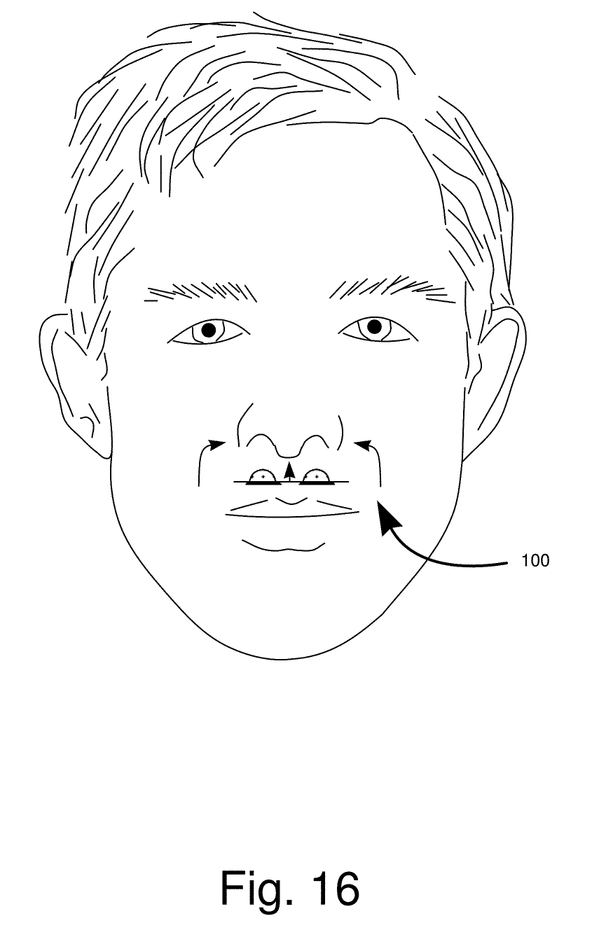

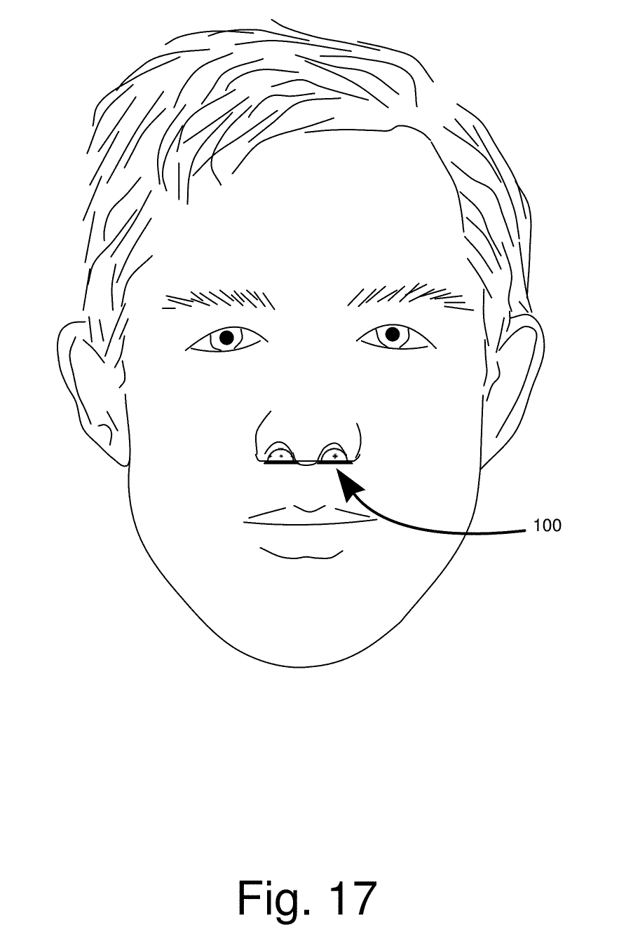

FIG. 16 is a frontal view of a face illustrating application of an embodiment of the nasal delivery device 200. FIG. 17 is a frontal view of a face illustrating the double nasal delivery device 200 attached to the nose. In the illustrated embodiment, the nasal delivery device 200 is situated so that the fabric domes 210 will cover both nostrils. The adhesive of the flexible base strip 230 is utilized to secure the nasal opening strip to the septum and to the nasal tissue surrounding the nostrils. The nasal delivery device 200 is shown merely as one illustration of the method and appearance of the various disclosed nasal delivery devices 100, 200, 300, 400, and 500 as attached to a nose. It should be noted to a person of ordinary skill in the art that any of the herein disclosed devices could be substituted into the above illustration.

Based on the foregoing, it should be appreciated that various concepts and technologies for providing various nasal delivery devices 100, 200, 300, 400, and 500 respectively, have been presented herein. The nasal delivery devices respectively include fabric pads or fabric domes positioned lengthwise along a center section of a flexible base strip. The flexible base strip also includes an adhesive layer affixed to the side of the flexible base strip that is used to attach the nasal delivery device to the nose.

### Example 1

The effects of various fabric samples on the humidity levels delivered to the respiratory tract were assessed in the laboratory using a mechanical ventilator and fabrics placed in a cylindrical, acrylic sample chamber. The ventilator was set to deliver 500 mL air volumes at a rate of 10 L/minute, with room air drawn in through the fabric sample during the inhale portion of the ventilation cycle and high humidity (95%-98%) air pumped out through the fabric sample during the exhale part of the cycle. These values approximate those of human breath during sleep.

The sample holder was designed to allow placement of a 1.9 cm diameter (˜2.8 cm² surface area) fabric sample over the end of a 1.9 cm diameter×5.7 cm long cylindrical chamber. The volume of the sample chamber (16.4 mL) was designed to approximate the average volume of the human nasal cavity (17.7 mL; Trindade, Inge, et. al., “Adult nasal volumes assessed by acoustic rhinometry,” Rev. Bras. Otorrinolaringol., Vol. 73, no. 1, 2007). The inside of the chamber was fitted with an automated hygrometer (Reed Instruments, Wilmington, N.C.; model SD-3007) set to record humidity readings at 2-second intervals.

Using this system, tests were conducted with various fabric samples in which the humidity levels during the inhale portion of the breathing cycle were measured with the humidity probe placed inside the sample chamber and positioned 1 cm behind the fabric sample. These levels were compared to the humidity levels measured with the hygrometer probe in the same position, but with no fabric sample in place. Tests were conducted in a laboratory where (due to seasonal variations) the humidity levels varied in different tests from a low of ˜30% to a high of ˜60%. The tests were initiated with the humidity probe mounted in the empty (i.e., no fabric installed) sample chamber, and data was collected for approximately 5 minutes. At that point the fabric sample was installed in the sample chamber and data collection was resumed. For most tests, data was collected, with the fabric sample installed, for between 15 and 30 minutes. In other instances data was collected for up to 5 hours (see EXAMPLE 2 below).

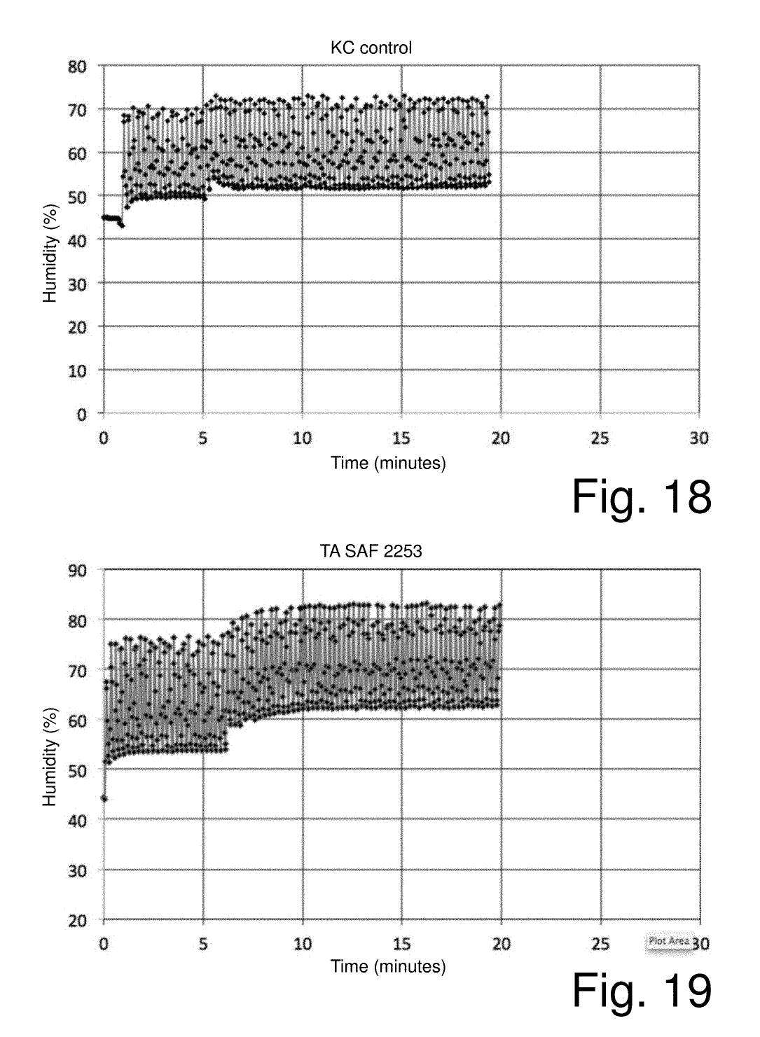

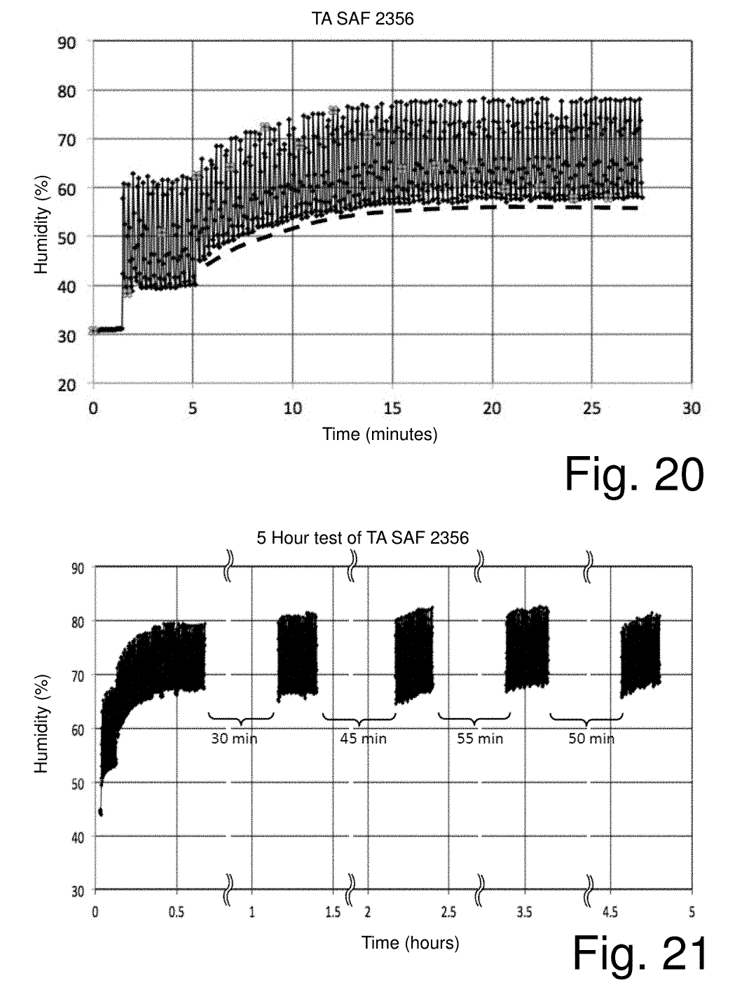

Results from tests conducted for approximately 20 minutes for three fabrics are shown in FIG. 18, FIG. 19, and FIG. 20. The fabric labeled “KC control” (see FIG. 18) is a through-air bonded, carded-web nonwoven (code #MGL9) supplied by Kimberly Clark Corp. (Neenah, Wis.), with a thickness of ˜4 mm and a basis weight of 100 gsm. The fabric labeled TA SAF 2253 (see FIG. 19) is an airlaid fabric that contains superabsorbent (SA) fibers (supplied by Technical Absorbents, Grimsby, England), with a thickness of ˜2 mm and a basis weight of ˜120 gsm. The fabric labeled TA SAF 2356 (see FIG. 20) is a needlefelt fabric that also contains superabsorbent (SA) fibers (supplied by Technical Absorbents), with a thickness of 1.9 mm and a basis weight of 140 gsm. This material contains approximately 70% superabsorbent fiber by weight. The graphs in FIG. 18, FIG. 19, and FIG. 20, display a band of data at each time point, showing the spread in humidity levels during each breathing cycle. The low end of the data band represents the measured inhale humidity at a given time point. The dotted line in the TA SAF 2356 curve highlights the gradual increase in inhaled humidity for this material.

Each of the three fabrics tested were equilibrated at the room humidity level overnight before testing. As shown in the table below, the KC control fabric produced a negligible increase in the humidity of the inhaled air. Inhale humidity readings with no sample in place (50%) increased by a modest 2% to 52% after the fabric was installed in the sample holder. The TA SAF 2253 fabric that contains superabsorbent fiber produced an increase in the humidity of the inhaled air from 54% to 63%. This represents an increase of 9 points on the humidity scale, or a total increase in the humidity level of 17%. The TA SAF 2356 fabric produced an increase in the humidity of the inhaled air from 40% to 58%. This represents an increase of 18 points on the humidity scale, or a total increase in the humidity level of 45%. TA SAF 2356 fabric samples have shown the highest increases in inhale humidity levels of the materials tested to date, with increases ranging from 27% to 51%, depending upon the sample and the starting room humidity.

TABLE 1
Example 1 humidity changes measured
for 3 different fabric samples.

| | Humidity level | | | |
|---|---|---|---|---|
| Test fabric | No sample | With sample | Humidity Δ | % Increase |
| KC control | 50% | 52% | 2% | 4% |
| TA 2253 | 54% | 63% | 9% | 17% |
| TA 2356 | 40% | 58% | 18% | 45% |

### Example 2

As discussed in EXAMPLE 1, the tests of TA SAF 2356 demonstrated that inhale humidity levels with this fabric that contains superabsorbent fiber tend to increase over the initial 15 minutes of testing, but then stabilize. Using the test system described in EXAMPLE 1, a test of TA SAF 2356 was conducted for approximately 5 hours to determine if inhale humidity levels would continue to be stable. The TA SAF 2356 fabrics were equilibrated at the laboratory humidity level overnight before testing. For these tests the mechanical ventilator remained in operation for the entire 5-hour period, but humidity measurement were taken for periods ranging from 15 to 45 minutes at intervals of 30 to 55 minutes.

Results for one of these tests are shown in FIG. 21. For this test the initial room humidity was approximately 45%. The empty chamber inhale humidity level at the beginning of the test was approximately 53%. With the TA SAF 2356 test material in place the inhale humidity level increased quickly and stabilized at approximately 68%. This represents an increase of 15 points on the humidity scale or an increase in the humidity level versus the empty chamber of 28%. (This also represents an increase in the inhale humidity level compared to the starting room humidity of 23 points, a 51% increase.) Subsequent inhale humidity readings over the course of the 5-hour test varied slightly from this initial stable value, but appeared to correspond to small changes in the room humidity that occurred during the test.

### Example 3

The effects on inhale humidity levels produced with fabric coated with humectant solutions were tested. For these tests the KC control nonwoven fabric described in EXAMPLE 1 was used. These tests were conducted with both dry samples and with samples that had been pre-humidified.

Humectant coated fabric samples were prepared as follows. Sample discs (19 mm diameter) were cut from sheets of KC through-air bonded, carded-web nonwoven (code #MGL9). Humectant solution was prepared by dissolving 100 mg of guar gum (Bob's Red Mill; Milwaukie, Oreg.) in 80 mL of a 20% sorbitol solution (Geritrex LLC, Mount Vernon, N.Y.). Sorbitol (a sugar alcohol) is the humectant in this solution and guar gum acts as a thickening agent. Discs of KC nonwoven fabrics were coated with the humectant solution using a variation of a standard “dip and nip” procedure. Sample discs were submerged in the humectant solution for 1 minute, removed and then allowed to drain for 1 minute. Excess humectant solution was removed by placing the wetted and drained sample discs on 8 layers of paper towel on a level surface and a second 8-layer stack of paper towel was placed on top of the wetted and drained sample discs. Pressure (˜15 psi) was then applied to the paper towel stack for 1 minute. The sample discs were then removed and allowed to air-dry overnight in a dust free environment.

Three samples were tested: a KC nonwoven control, KC nonwoven treated with humectant, and KC nonwoven treated with humectant and “moisturized.” A KC fabric control disc and a humectant-treated KC fabric disc were equilibrated against the room humidity overnight in an open, dust-free container. A second humectant-treated KC fabric disc was “moisturized” overnight in a sealed plastic container that contained a wetted pad of paper towel not in direct contact with the humectant-treated KC fabric disc.

All three samples were tested consecutively on the same day using the protocol outlined in EXAMPLE 1. Results are shown in the table below. As seen in EXAMPLE 1, the KC control fabric produced no significant elevation in the inhale humidity level. In contrast, both the humidified and the non-humidified humectant-coated KC fabrics induced substantial increases in inhale humidity. The one small difference between the humidified and non-humidified fabrics was that the humidified sample reached the elevated inhale humidity levels within approximately 2 minutes; the non-humidified sample reached a stable inhale humidity level in approximately 12 minutes.

TABLE 3
Example 3 humidity changes measured
for 3 different fabric samples.

| | Humidity level | | | |
|---|---|---|---|---|
| Test fabric | No sample | With sample | Humidity Δ | % Increase |
| KC control | 44% | 46% | 2% | 4% |
| KC + humectant | 45% | 61% | 16% | 36% |
| Humidified | 46% | 62% | 16% | 35% |
| KC + humectant | | | | |

### Example 4

The effects on inhale humidity levels produced with fabric containing superabsorbent fiber were tested with and without pre-humidification. Three fabric samples were tested: a KC nonwoven control, a TA SAF 2356 fabric sample, and a “moisturized” TA SAF 2356 fabric sample. A KC fabric control disc and a non-moisturized TA SAF 2356 fabric disc were equilibrated against room humidity overnight in an open, dust-free container. As described in EXAMPLE 3, the TA SAF 2356 fabric sample was pre-moistened overnight in a sealed plastic container that contained a wetted pad of paper towel not in direct contact with the TA SAF 2356 fabric disc.

All three samples were tested consecutively on the same day using the protocol outlined in EXAMPLE 1. Results are shown in the table below. As described in EXAMPLE 1, the KC control fabric produced no significant elevation in the inhale humidity levels. In contrast, and similar to the results shown in EXAMPLE 3, both the humidified and the non-humidified TA SAF 2356 fabrics induced substantial increases in inhale humidity. Again, the one slight difference between the humidified and non-humidified fabrics is that the humidified sample reached the elevated inhale humidity levels quicker, within approximately 2 minutes; the non-humidified sample reached a stable inhale humidity level in approximately 15 minutes.

TABLE 4
Example 4 humidity changes measured
for 3 different fabric samples.

| | Humidity level | | | |
|---|---|---|---|---|
| Test fabric | No sample | With sample | Humidity Δ | % Increase |
| KC control | 41% | 42% | 1% | 2% |
| TA SAF 2356 | 44% | 68% | 24% | 55% |
| Humidified | 43% | 66% | 23% | 54% |
| TA SAF 2356 | | | | |

### Example 5

A final example is presented to demonstrate the ability of the present invention to deliver volatile compounds to the respiratory system. In this experiment a KC nonwoven control fabric sample disc was coated with menthol. Room air was then passed through the sample and collected in a hexane solution that was assayed for menthol content using GC-FID (gas chromatograph with flame ionization detector) instrumentation.

Menthol is a waxy, crystalline solid at room temperature that becomes a liquid at 31° C. A menthol solution was prepared to deposit a known amount of menthol onto a 1.9 cm diameter KC control fabric disc. 0.25 g of menthol crystals (Mentha Arvensis; Dreaming Earth Botanicals, Asheville, N.C.) was dissolved in 10 mL of chromatography-grade hexane and 1 mL of this solution was deposited slowly onto the KC control fabric disc, insuring that all the solution remained in the fabric web. The hexanes were then allowed to evaporate leaving menthol crystals deposited on the fabric fibers. The menthol-treated fabric disc was then loaded into the acrylic sample holder described in EXAMPLE 1.

Using the mechanical ventilator, room air was passed through the treated fabric sample and 50 500 mL “breaths” (total volume 25 L) were passed through the sample and into the hexane solution in a fume hood. The expelled air, after traveling across the sample, passed through a tube connected to a diffuser stem that was immersed in 120 mL of hexane in a graduated cylinder. This hexane collection solution contained 10 mM eugenol (Sigma-Aldrich Corp., St. Louis, Mo.) that acted as an internal standard during the GC-FID analysis.

During the passage of 25 L of room air through the sample and into the hexane/eugenol solution, the volume of hexane/euginol solution decreased from 120 mL to 84 mL due to evaporation. 1 mL of the collected solution was analyzed on the GC-FID instrument and compared to menthol standards prepared in the same hexane/eugenol solution. The concentration of menthol in the collection volume was measured at 40.56 mM. This translates to 6.34 mg of menthol in the 84 mL of collection fluid. These results indicate that a minimum of 0.16% of the deposited menthol on the fabric sample was released and collected in the hexane collection fluid. This is a minimum estimate; clearly much of the air passed through the menthol-coated sample and through the hexane solution may not have transferred the menthol vapor to the collection fluid. These results demonstrate that volatile compounds can be delivered to the respiratory system using the present invention.

The subject matter described above is provided by way of illustration only and should not be construed as limiting. Furthermore, the claimed subject matter is not limited to implementations that solve any or all disadvantages noted in any part of this disclosure. Various modifications and changes may be made to the subject matter described herein without following the example embodiments and applications illustrated and described, and without departing from the true spirit and scope of the present invention, which is set forth in the following claims.

What is claimed is:

1. A inhalation therapy device for providing moisture into nasal passages of an individual, the inhalation therapy device comprising:
a fabric pad that includes a breathable fabric layer having at least one type specified fiber for collecting moisture;
a base strip for attachment to nostril openings of a nose, the base strip having sufficient flexibility for adapting to the nose shape, and having openings corresponding to the nostril openings, the base strip having a skin side and a fabric side, the skin side including an adhesive for securing the base strip to the nose; and
a double-sided adhesive for affixing the fabric pad to the fabric side of the base strip,
wherein the fabric pad absorbs moisture from exhaled air and provides that moisture into the nasal passages during inhalation.

2. The inhalation therapy device of claim 1, the breathable fabric layer further comprising sodium polyacrylate fibers.

3. The inhalation therapy device of claim 1, the breathable fabric layer further comprising humectants-coated fibers.

4. The inhalation therapy device of claim 1, the breathable fabric layer further comprising synthetic hygroscopic fibers.

5. The inhalation therapy device of claim 1, the breathable fabric layer further comprising plant-based hygroscopic fibers.

6. The inhalation therapy device of claim 1, the breathable fabric layer further comprising animal-based hygroscopic fibers.

7. The inhalation therapy device of claim 1, wherein the breathable fabric layer includes a coating of volatile compounds.

8. The inhalation therapy device of claim 7, the volatile compounds further comprising at least one of:
cough suppressant;
essential oils;
aromatherapy agents; and
respiratory medication.

9. The inhalation therapy device of claim 1, wherein the fabric pad includes two parts corresponding respectively to each nostril opening, and the double-sided adhesive includes two parts corresponding respectively to the two parts of the fabric pad.

10. The inhalation therapy device of claim 9, wherein the two parts of the fabric pad are respective domes that protrude through each respective nostril opening of the base strip.

11. A inhalation therapy device for providing moisture into nasal passages of an individual, the inhalation therapy device comprising:
a fabric pad that includes a breathable fabric layer coated with volatile compounds;
a base strip for attachment to nostril openings of a nose, the base strip having sufficient flexibility for adapting to the nose shape, and having openings corresponding to the nostril openings, the base strip having a skin side and a fabric side, the skin side including an adhesive for securing the base strip to the nose; and
a double-sided adhesive for affixing the fabric pad to the fabric side of the base strip,
wherein the fabric pad absorbs moisture from exhaled air and provides that moisture into the nasal passages during inhalation.

12. The inhalation therapy device of claim 11, the volatile compounds further comprising at least one of:
cough suppressant;
essential oils;
aromatherapy agents; and
respiratory medication.

13. The inhalation therapy device of claim 11, the breathable fabric layer further comprising humectants-coated fibers.

14. The inhalation therapy device of claim 11, the breathable fabric layer further comprising synthetic hygroscopic fibers.

15. The inhalation therapy device of claim 11, the breathable fabric layer further comprising plant-based hygroscopic fibers.

16. The inhalation therapy device of claim 11, the breathable fabric layer further comprising animal-based hygroscopic fibers.

17. A inhalation therapy device for providing moisture into nasal passages of an individual, the inhalation therapy device comprising:
an adhesive strip for attachment to at least one nostril opening, the adhesive strip having sufficient flexibility for adapting to nose shape, and having at least one opening corresponding to the at least one nostril opening; and
a fabric pad affixed to the adhesive strip, the fabric pad having a breathable fabric layer that includes sodium polyacrylate fibers for collecting moisture,
wherein the fabric pad absorbs moisture from exhaled air and provides that moisture into at least one nasal passage during inhalation.

18. The inhalation therapy device of claim 17, further comprising:
the fabric pad having two parts corresponding respectively to two nostril openings; and
the adhesive strip having two openings corresponding respectively to the two parts of the fabric pad.

19. The inhalation therapy device of claim 17, wherein the adhesive strip has openings corresponding to the at least one nostril opening, and is a double-sided adhesive having a fabric pad side for securing the fabric pad to the adhesive strip and a skin-sensitive side for securing the inhalation therapy device to a nose.

20. The inhalation therapy device of claim 17, the adhesive strip further comprising a single opening corresponding to a single nostril opening, and a skin-sensitive adhesive for securing the inhalation therapy device to a nose, and the inhalation therapy device further comprising one of the following:
a single fabric pad corresponding to the single nostril;
a single fabric dome corresponding to the single nostril.
